(19) United States
(12) Patent Application Publication
(10) Pub. No.: US 2026/0243719 A1
(43) Pub. Date: Aug. 20, 2026

(54) SENSOR DEVICE FOR MULTI-TYPE ION DETECTION AND MANUFACTURING METHOD THEREOF

(71) Applicant: KOREA INSTITUTE OF SCIENCE AND TECHNOLOGY, Seoul (KR)

(72) Inventors: Yi-Jae Lee, Seoul (KR); Yamujin Jang, Seoul (KR)

(73) Assignee: KOREA INSTITUTE OF SCIENCE AND TECHNOLOGY, Seoul (KR)

(21) Appl. No.: 19/282,045

(22) Filed: Jul. 28, 2025

(30) Foreign Application Priority Data
Feb. 14, 2025 (KR) 10-2025-0019580

(51) Int. Cl. G01N 27/12

(52) U.S. Cl. CPC G01N 27/125

(57) ABSTRACT

Provided are a sensor device for multi-type ion detection and a manufacturing method thereof. Provided are a sensor device for multi-type ion detection including a wafer substrate, and an array of a sensor chip arranged on the wafer substrate, in which the sensor chip includes a plurality of line sensors, and the line sensor includes source, drain, and gate electrodes formed on the wafer substrate, a conductive polymer channel layer formed on the wafer substrate between the source and drain electrodes, and a sensing unit formed on the conductive polymer channel layer.

### CROSS-REFERENCE TO RELATED APPLICATION

This application claims the benefit of Korean Patent Application No. 10-2025-0019580, filed on Feb. 14, 2025, in the Korean Intellectual Property Office, the entire disclosure of which is incorporated herein by reference for all purposes.

### BACKGROUND

### 1. Field of the Invention

One or more embodiments relate to a sensor device for multi-type ion detection and a manufacturing method thereof.

### 2. Description of the Related Art

Among biochemical sensors, ion sensors detect specific ions present in a solution and display a potential corresponding to a concentration and activity of the ions. Recently, the importance of high-sensitivity and multi-ion measurement technology has been highlighted in small and lightweight environmental and healthcare systems (e.g., water quality testing, disease diagnosis, sports/military use, etc.).

Generally, ion detection methods using ion chromatography are used, and recently, electrochemical-based ion sensors have been commercialized. Most of these are probe-type, which limits miniaturization and makes them unsuitable as platforms for simultaneous detection of multiple ions in saliva.

Therefore, it is necessary to develop an ultra-small and low-voltage multi-type ion detection sensor based on organic electronic materials that may be used in technologies of wearable platforms for healthcare and fixed multi-type ion detection platforms.

### SUMMARY

Embodiments provide a sensor device for multi-type ion detection, which is capable of simultaneously detecting sugar (glucose) and pH as well as detecting multi-type ions in a sample with ultra-small size and low-power operation. For example, embodiments provide a sensor device for detecting multi-type ions, which are a wafer-level chip platform configured with a sensor chip array based on organic electronic materials.

Embodiments provide a method of manufacturing a sensor device for multi-type ion detection according to embodiments of the present disclosure.

However, technical goals to be achieved are not limited to those described above, and other goals not mentioned above can be clearly understood by one of ordinary skill in the art from the following description.

According to an aspect, there is provided a sensor device for multi-type ion detection including a wafer substrate, and an array of a sensor chip arranged on the wafer substrate, wherein the sensor chip includes a plurality of line sensors, and the line sensor includes source, drain, and gate electrodes formed on the wafer substrate, and a sensing unit including a conductive polymer channel layer formed on the wafer substrate between the source and drain electrodes.

An upper end of the conductive polymer channel layer may extend to a portion of the upper end of the source and drain electrodes, and the conductive polymer channel layer may have a thickness of 1 micrometer (μm) or less.

The conductive polymer channel layer may include dimethyl sulfoxide (DMSO)-modified poly(3,4-ethylenedioxythiophene) polystyrene sulfonate (PEDOT: PSS), ethylene glycol (EG)-modified PEDOT: PSS, or both.

The sensor chip may detect ions in a body fluid or saliva, and the plurality of line sensors in the sensor chip may have different detection targets.

The sensor chip may detect at least two or more ions among K⁺ ions, Na⁺ ions, Ca⁺ ions, and Cl⁻ ions in a body fluid or saliva.

The sensor chip may further include a function of detecting glucose in blood and a function of measuring a pH of a solution.

The sensing unit may further include an ion-selective membrane layer formed on the conductive polymer channel layer.

The sensing unit may further include a well, the conductive polymer channel layer may be exposed on a bottom surface of the well, or an ion-selective membrane layer may be formed on the exposed conductive polymer channel layer.

Some of the plurality of line sensors in the sensor chip may include the sensing unit including the conductive polymer channel layer, and the remaining line sensors may include the sensing unit including the conductive polymer channel layer and the ion-selective membrane layer, or all of the plurality of line sensors in the sensor chip may include the sensing unit including the conductive polymer channel layer and the ion-selective membrane layer.

The ion-selective membrane layer may include ionophores, and some or all of the plurality of line sensors may include different ionophores.

The ionophores may include at least one of valinomycin, potassium salt of tetrakis(4-chloro-phenyl)borate (KTCIPB), Na Ionophore X, sodium tetrakis[3,5-bis(trifluoromethyl)phenyl]borate (Na-TFPB), N,N,N′,N′-tetra[cyclohexyl]diglycolic acid diamide, N,N,N′,N′-Tetracyclohexyl-3-oxapentanediamide (ETH129), NaTFPB, 3,6-didodecyloxy-4,5-dimethyl-o-phenylene-bis(mercury chloride) (ETH9033), and tridodecylmethylammonium chloride (TDMACl).

When the ionophores include two or more types, a mass ratio (w/w) of one ionophore to the remaining ionophores may be 1:0.1 to 3.

The ion-selective membrane layer may include K ⁺ionophores including valinomycin and KTCIPB.

The ion-selective membrane layer may include Na ⁺ionophores including Na Ionophore X and Na-TFPB.

The ion-selective membrane layer may include Ca²⁺ ionophores including ETH129 and NaTFPB.

The ion-selective membrane layer may include Cl⁻ ionophores including ETH9033 and TDMACl.

The ion-selective membrane layer may include a polyaniline (PANi)-polyvinyl chloride (PVC) gel for pH measurement.

According to another aspect, there is provided a method of manufacturing a sensor device for multi-type ion detection, the method including preparing a wafer substrate, and forming an array of a sensor chip including a plurality of line sensors on the wafer substrate, wherein the forming of the array of the sensor chip includes forming source, drain, and gate electrodes on the wafer substrate, and forming a sensing unit, the forming of the sensing unit includes forming a conductive polymer channel layer on the wafer substrate between the source and drain electrodes, and the sensor device is the sensor device described above.

The forming of the sensing unit may include forming the conductive polymer channel layer on the wafer substrate between the source and drain electrodes, and forming an ion-selective membrane layer on the conductive polymer channel layer, and the ion-selective membrane layer may be formed by drop casting or spin coating.

The forming of the sensing unit may include patterning a well after applying a polymer film on the conductive polymer channel layer, and the conductive polymer channel layer may be exposed to a bottom surface of the well.

Additional aspects of embodiments will be set forth in part in the description which follows and, in part, will be apparent from the description, or may be learned by practice of the disclosure.

The present disclosure may provide a sensor device for multi-type ion detection based on organic electronic materials for multi-type ion detection in a sample (e.g., saliva or a body fluid), and a manufacturing method thereof.

The present disclosure may provide a sensor device for multi-type ion detection based on organic electronic materials capable of miniaturization and low-power operation, and a manufacturing method thereof.

The present disclosure may provide a sensor device for wafer-level multi-type ion detection configured with an organic electronic material-based sensor chip or an array thereof, and a manufacturing method thereof.

According to an embodiment, the present disclosure may provide a sensor device for multi-type ion detection based on organic electronic materials for detecting multi-type ions such as K, Na, Ca, Cl, and the like in saliva with high selectivity and high sensitivity, and a manufacturing method thereof.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 illustrates an example of a configuration of a sensor device for wafer-level multi-type ion detection according to an embodiment;

FIG. 2 illustrates an example of a configuration of a sensor chip of a sensor device according to an embodiment;

FIGS. 3A, 3B, 3C, and 3D illustrate examples of a configuration of a sensing unit of a sensor chip according to an embodiment;

FIG. 4 illustrates an example of a manufacturing process of a sensor device for wafer-level multi-type ion detection according to an embodiment;

FIG. 5 illustrates an example of a manufacturing process of a sensor device for wafer-level multi-type ion detection according to an embodiment;

FIG. 6 illustrates an optical microscope (OM) image after forming an ion-selective membrane for sensing four types of ions (K, Na, Ca, and Cl) in a sensing device for wafer-level multi-type ion detection manufactured in examples;

FIG. 7 illustrates an OM image after forming a polyaniline (PANi) membrane subjected to drop casting (drop coating) for pH sensing in a sensor device for wafer-level multi-type ion detection manufactured in examples;

FIG. 8 illustrates an OM image after forming poly(3,4-ethylenedioxythiophene) polystyrene sulfonate (PEDOT: PSS) for blood glucose sensing in a sensor device for wafer-level multi-type ion detection manufactured in examples;

FIG. 9 illustrates K⁺ ion sensing characteristics of a sensor device for wafer-level multi-type ion detection manufactured in examples;

FIG. 10 illustrates Na⁺ ion sensing characteristics of a sensor device for wafer-level multi-type ion detection manufactured in examples;

FIG. 11 illustrates Ca⁺ ion sensing characteristics of a sensor device for wafer-level multi-type ion detection manufactured in examples;

FIG. 12 illustrates Cl⁻ ion sensing characteristics of a sensor device for wafer-level multi-type ion detection manufactured in examples;

FIG. 13 illustrates pH sensing characteristics of a sensor device for wafer-level multi-type ion detection manufactured in examples;

FIG. 14 illustrates sugar sensing characteristics of a sensor device for wafer-level multi-type ion detection manufactured in examples;

FIG. 15 illustrates K⁺ ion sensing results in a standard solution and artificial saliva of a sensor device for wafer-level multi-type ion detection manufactured in examples;

FIG. 16 illustrates Na⁺ ion sensing results in a standard solution and artificial saliva of a sensor device for wafer-level multi-type ion detection manufactured in examples;

FIG. 17 illustrates Ca⁺ ion sensing results in a standard solution and artificial saliva of a sensor device for wafer-level multi-type ion detection manufactured in examples;

FIG. 18 illustrates Cl⁻ ion sensing results in a standard solution and artificial saliva of a sensor device for wafer-level multi-type ion detection manufactured in examples;

FIG. 19 illustrates low-voltage operation characteristics according to a channel material of a sensor device for wafer-level multi-type ion detection manufactured in examples; and

FIG. 20 illustrates low-voltage operation characteristics according to voltages of source and drain electrodes of a sensor device for wafer-level multi-type ion detection manufactured in examples.

### DETAILED DESCRIPTION

Hereinafter, embodiments of the present disclosure will be described in detail with reference to the accompanying drawings. In the following description of the embodiments, a detailed description of known functions and configurations incorporated herein will be omitted when the same may make the subject matter of the embodiments disclosed in the present specification rather unclear. In addition, terminologies used herein are defined to appropriately describe the embodiments and thus may be changed depending on a user, the intent of an operator, or a custom of a field to which the present disclosure pertains. Accordingly, the terminologies must be defined based on the following overall description of the present specification. In the drawings, like reference numerals are used for like elements.

In the whole specification, when a member is positioned “on” another member, this not only includes a case that the any member is brought into contact with the other member, but also includes a case that another member exists between two members.

It will be understood that when a certain part “includes” a certain component, the part does not exclude another component but may further include another component.

As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof. Terms such as “1st” and “2nd,” or “first” and “second” may be used to simply distinguish a corresponding component from other components, and do not limit the components in other aspects (e.g., importance or order). For example, suitable results may be achieved if the described techniques are performed in a different order and/or if components in a described system, architecture, device, or circuit are combined in a different manner and/or replaced or supplemented by other components or their equivalents. Therefore, other implementations, other embodiments, and equivalents to the claims are also within the scope of the following claims.

Hereinafter, a sensor device for multi-type ion detection and a manufacturing method thereof will be described in detail with reference to the embodiments and the drawings. However, the present disclosure is not limited to the embodiments and drawings.

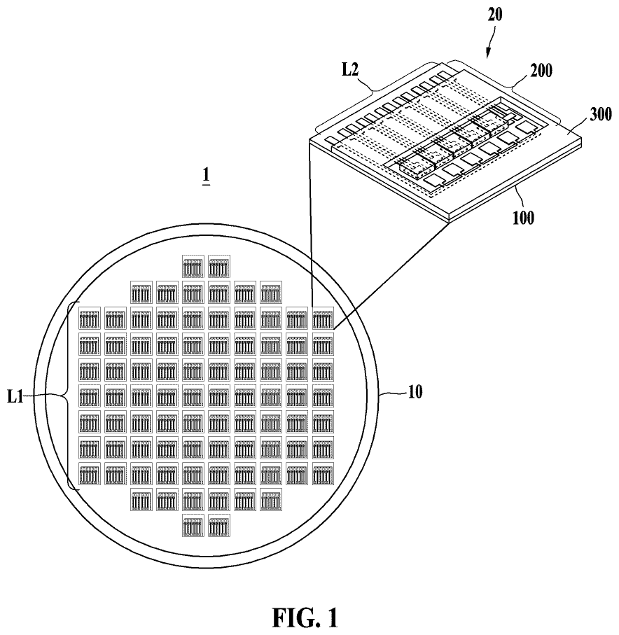

According to an embodiment, the present disclosure provides a sensor device for wafer-level multi-type ion detection. Referring to FIG. 1, FIG. 1 illustrates an example of a configuration of a sensor device 1 for multi-type ion detection according to embodiments of the present disclosure, and in FIG. 1, the sensor device 1 for multi-type ion detection may be a wafer-level sensor device including a sensor chip array L1 in which a plurality of sensor chips 20 are arranged on a substrate 10. The sensor device 1 may implement multi-sensing capable of sensing multi-type ions simultaneously or sensing multi-type ions, sugars, and pH simultaneously in one sample or a plurality of samples (multiple samples) by using a sensor chip array. The multi-sensing may be performed at the sensor chip and/or sensor device level. In addition, the sample may be selected from saliva, body fluids (e.g., blood), and solutions (e.g., water). The substrate 10 is a semiconductor substrate, and may be a wafer substrate, a silicon substrate, a sapphire substrate, or the like, but is not limited thereto.

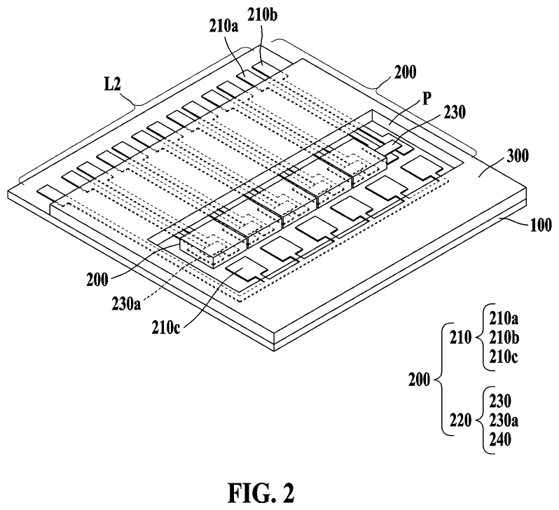

According to an embodiment, referring to FIG. 2, FIG. 2 illustrates an example of a configuration of the sensor chip 20 for multi-type ion detection according to embodiments of the present disclosure. In FIG. 2, the sensor chip 20 may include a line sensor array L2 in which a plurality of line sensors 200 are arranged on a substrate 100 (e.g., the substrate 10 of FIG. 1), and a cover portion 300 that covers a portion of the line sensor array L2 and has an opening area P. Each line sensor 200 may include an electrode 210 (e.g., 210a, 210b, and 210c) formed on the substrate 100, and a sensing unit 220. The sensor chip 20 is an organic electronic material-based sensor that may implement multi-sensing capable of sensing multi-type ions simultaneously or sensing multi-type ions, sugars, and pH simultaneously in one sample or a plurality of samples (multiple samples).

According to an embodiment, the substrate 100 may be applied without limitation as long as it is applicable to the sensor, and may be, for example, a wafer substrate, a silicon substrate, glass, a polymer substrate, a sapphire substrate, and the like. In addition, the substrate 100 may be the substrate 10 of the sensor device 1.

According to an embodiment, the electrode 210 may be configured as an electrode unit including a source electrode 210a, a drain electrode 210b, and a gate electrode 210c in one line sensor 200. A single sensor chip 20 may include an electrode array formed of a plurality of electrode units patterned to be connected to each other. The electrode unit may be applied to any material without limitation as long as it is a material that may be used as an electrode of a sensor device, and may include, for example, an organic or inorganic conductive material, a metal (e.g., chromium (Cr), gold (Au)), a metal alloy, and the like.

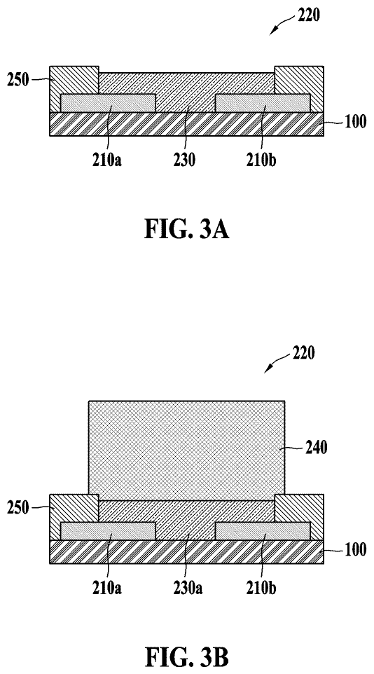

According to an embodiment, referring to FIGS. 3A, 3B, 3C, and 3D, FIGS. 3A, 3B, 3C, and 3D illustrate examples of a configuration of the sensing unit 220 according to the present disclosure, according to embodiments of the present disclosure. The sensing unit 220 may include a conductive polymer channel layer 230 or 230a, and may further include an ion-selective membrane layer 240 on the conductive polymer channel layer 230 or 230a. For example, the line sensor array L2 may include a sensing portion A including the conductive polymer channel layer 230 (e.g., FIGS. 3A and 3C), a sensing portion B including the conductive polymer channel layer 230a and the ion-selective membrane layer 240 formed on the conductive polymer channel layer 230a (e.g., FIGS. 3B and 3D), or both.

According to an embodiment, the conductive polymer channel layer 230 may be formed on the substrate 100 between the source electrode 210a and the drain electrode 210b. An upper end of the conductive polymer channel layer 230 may extend to a portion of an upper area of each of the source electrode 210a and the drain electrode 210b to cover a portion of each of the source electrode 210a and the drain electrode 210b. The conductive polymer channel layer 230 is formed of an organic electronic material and may provide low-power operation and high-sensitivity sensor performance. A thickness of the conductive polymer channel layer 230 may be about 1 micrometer (μm) or less, about 0.9 μm or less, about 0.8 μm or less, about 0.5 μm or less, or about 0.1 μm or less (or more), and the mentioned values may be minimum and/or maximum values. Alternatively, a root-mean-square (RMS) value of a surface roughness of the conductive polymer channel layer 230 may be about 2 nanometers (nm) to about 3 nm.

The thickness may be a distance from a lower end to an upper end of the conductive polymer channel layer 230 on the substrate 100. The conductive polymer channel layer 230 may include an organic conductive polymer. The organic conductive polymer may include at least one or two or more of dimethyl sulfoxide (DMSO)-modified poly(3,4-ethylenedioxythiophene) polystyrene sulfonate (PEDOT: PSS), ethylene glycol (EG)-modified PEDOT: PSS, and DMSO and EG-modified PEDOT: PSS. For example, the DMSO may modify PEDOT: PSS at 15% to 25% by volume with respect to PEDOT: PSS (based on a total volume). The EG may modify PEDOT: PSS at 1 wt % to 8 wt % with respect to PEDOT: PSS (based on a total mass).

According to an embodiment, all or a portion of the line sensor array L2 may include conductive polymer channel layers 230 and 230a of the same or different components. The reference numerals “230 and 230a” of the conductive polymer channel layers are provided to distinguish the configuration of the sensing unit 220 with or without the ion-selective membrane layer 240.

According to an embodiment, the sensing unit 220 may include the ion-selective membrane layer 240, and the ion-selective membrane layer 240 may be formed on the conductive polymer channel layer 230 so as to be in contact with the conductive polymer channel layer 230 or 230a. A thickness of the ion-selective membrane layer 240 may be about 1 μm or more, about 30 μm or more, about 40 μm or more, about 50 μm or more, about 1 μm to about 100 μm, about 30 μm to about 50 μm, or about 40 μm to about 50 μm. The ion-selective membrane layer 240 may include ionophores, and the ionophores may be included as a composition mixed with a base material for matrix formation. The ionophores may include ionophores for K⁺, Na⁺, Ca²⁺, Cl⁻, or pH sensing. The ionophores may include, for example, at least one or two or more of valinomycin, potassium salt of tetrakis(4-chloro-phenyl)borate (KTCIPB), Na Ionophore X (Selectophore™, e.g., 4-tert-Butylcalix[4]arene-tetraacetic acid tetraethyl ester), sodium tetrakis[3,5-bis(trifluoromethyl)phenyl]borate (Na-TFPB), N,N,N′,N′-Tetra[cyclohexyl]diglycolic acid diamide, N,N,N′,N′-Tetracyclohexyl-3-oxapentanediamide (ETH129), sodium tetrakis[3,5-bis(trifluoromethyl)phenyl]borate (NaTFPB), 3,6-didodecyloxy-4,5-dimethyl-o-phenylene-bis(mercury chloride) (ETH9033), and tridodecylmethylammonium chloride (TDMACl). The ionophores may be selected according to a sensing target (e.g., ion), and may include, for example, K⁺ ionophores including valinomycin and KTCIPB, Na⁺ ionophores including Na Ionophore X and Na-TFPB, Ca²⁺ ionophores including ETH129 and NaTFPB, and Cl⁻ ionophores including ETH9033 and TDMACl. When two or more types of the ionophores are included, a mass ratio of one ionophore to the remaining ionophores may be 1:0.1 to 3, 1:0.1 to 2.5, 1:0.1 to 2, 1:0.1 to 1.5, 1:0.1 to 0.8, 1:0.1 to 0.5, or 1:0.1 to 0.3 (mass ratio; w/w). The base material may include polyvinyl chloride (PVC) and di-2-ethylhexyl sebacate (DOS), a ratio thereof may be 1:1.5 to 2 (w/w), and the content of the base material may be 90 wt % to 99 wt %, 92 wt % to 98 wt %, or 95 wt % to 98 wt % of the composition (e.g., a composition containing the ionophores and the base material) (based on the total mass). By applying the ionophore configuration described above, multi-type ion sensing and high-sensitivity sensing performance may be provided.

According to an embodiment, the ion-selective membrane layer 240 may include a polyaniline (PANi)-polyvinyl chloride (PVC) gel for pH sensing. A volume ratio (v/v) of PANi to PVC may be 1:1 to 2, or 1:1.2 to 1.6.

According to an embodiment, a passivation layer 250 may be formed on a portion on (i.e., on an upper end of) each of the source electrode 210a and the drain electrode 210b where the conductive polymer channel layer 230 is not formed (i.e., not extended). That is, the passivation layer 250 may cover the area of the substrate 100 other than the electrode-patterned portion and may be disposed between the drain electrode 210b and the gate electrode 210c. The passivation layer 250 may prevent the ion-selective membrane layer 240 from coming into contact with the electrode.

According to an embodiment, the sensing unit 220 may include the conductive polymer channel layer 230, which may be used for sugar measurement (FIGS. 3A and 3D). The sensing unit 220 may be used for ion detection and pH measurement by forming the conductive polymer channel layer 230 and the ion-selective membrane layer 240 on the conductive polymer channel layer 230.

According to an embodiment, the sensing unit 220 may include a well W, and may include the conductive polymer channel layer 230a and the ion-selective membrane layer 240 in the well W. For example, on a bottom surface within the well W, a portion (e.g., a portion of an upper end surface) of the conductive polymer channel layer 230a may be exposed. For example, in the sensing unit 220, the inside of the well W may be filled with the ion-selective membrane layer 240, and the ion-selective membrane layer 240 may come into contact with the conductive polymer channel layer 230a exposed within the well W (FIGS. 3C and 3D).

According to an embodiment, the cover portion 300 may be a well-shaped plate having the opening area P in which the sensing unit 200 is exposed to enable the sensing operation. The cover portion 300 may have the same or different components as the well W of the sensing unit 220. For example, the components thereof may be polydimethicone (PDMS), polystyrene, or polyurethane.

According to an embodiment, the plurality of line sensors 200 in the sensor chip 20 may set the configuration of the sensing unit according to a detection target, and may be configured as, for example, a line sensor for multi-type ion detection, a line sensor for sugar measurement, and a line sensor for pH measurement. Also, the number thereof may be set in various ways. In the sensor chip 20, the plurality of line sensors 200 may include the sensing unit 220 including different ionophores for multi-type ion detection. In addition, the line sensor 200 including the sensing unit 220 of the same configuration may be configured as a single line sensor or in plurality in a duplicate manner.

According to an embodiment, some or all of the plurality of line sensors 200 in the sensor chip 20 may include the sensing unit 220 including the ion-selective membrane layer 240.

According to an embodiment, some of the plurality of line sensors in the sensor chip 20 may include the sensing unit 220 including the ion-selective membrane layer 240, and the remaining line sensors may include the sensing unit 220 formed with the conductive polymer channel layer 230 without including the ion-selective membrane layer 240.

According to an embodiment, the sensor chip 20 detects ions in a body fluid or saliva, and may simultaneously detect two or more types of ions, three or more types of ions, or four or more types of ions. For example, the sensor chip 20 may detect at least two or more types, three or more types, or four or more types of K ions, Na ions, Ca ions, and Cl ions in saliva. For such ion detection, two or more line sensors, three or more line sensors, four or more line sensors, five or more line sensors, or six or more line sensors may be included based on the detection target. In addition, the sensor chip 20 may further have a function of measuring sugar (glucose) in saliva or a body fluid (e.g., blood) and measuring the pH of saliva, a body fluid, or a solution (e.g., water).

According to an embodiment, the line sensor array L2 in the sensor chip 20 may be configured as the line sensor array L2 including a line sensor for ion detection and optionally including an OTEC line sensor for sugar measurement, a line sensor for pH detection, or both.

According to an embodiment, the sensor chip 20 may include the line sensor array L2 configured with the plurality of line sensors 200, and at least some or all of the plurality of line sensors 200 within the sensor chip 20 may have different detection targets or components. For example, the sensor chip 20 may be configured with the line sensors 200 with different detection targets. For example, the sensor chip 20 may include some or all of the plurality of line sensors 200 capable of detecting ions. For example, in the sensor chip 20, some of the plurality of line sensors 200 are line sensors capable of detecting ions, and the remaining line sensors may include line sensors capable of detecting sugar, line sensors capable of detecting pH, or both.

According to an embodiment, a method of manufacturing the sensor device 1 of the present disclosure may implement the sensor chip array L1 by manufacturing the plurality of sensor chips 20 simultaneously on the substrate 100. The manufacturing method may include a step of preparing a substrate, and a step of forming a sensor chip array including a plurality of line sensors on the substrate. The substrate may be as described above for the sensor device 1 and may be, for example, a wafer substrate.

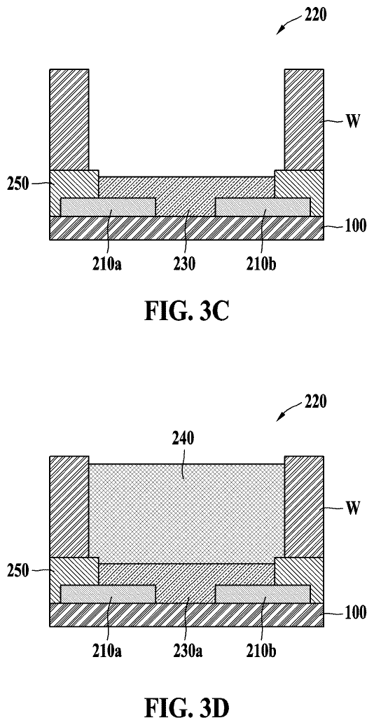

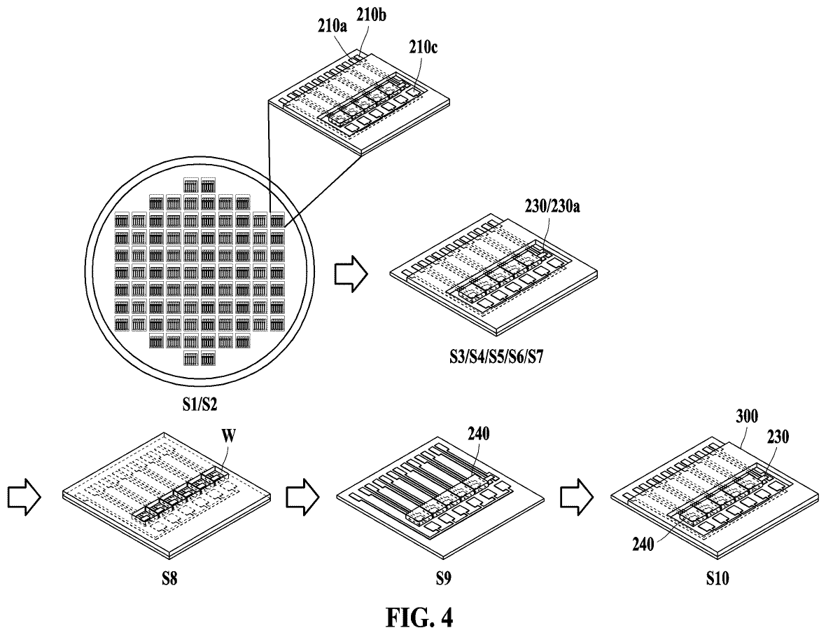

Referring to FIGS. 4 and 5, FIGS. 4 and 5 illustrate examples of a process of a method of manufacturing the sensor device 1 according to embodiment of the present disclosure, the step of forming the sensor chip array in FIGS. 4 and 5 may include forming a line sensor array in which a plurality of line sensors are arranged in each sensor chip, and may include a step of forming an electrode on the substrate (S1 and S2), a step of forming a sensing unit (S3 to S9), and a step S10 of forming a cover.

More specifically, the step of forming the sensor chip array may include a step of forming source, drain, and gate electrodes on the substrate (S1 and S2), a step of forming a passivation layer (S3, S4, and S5), a step of forming a conductive polymer channel layer (S6 and S7), and a step of forming an ion-selective membrane layer (S8 and S9).

According to an embodiment, the step of forming the electrodes (S1 and S2) may include forming a pattern film for electrode patterning on the substrate and removing the pattern film after depositing the electrodes. For example, a pattern film of PR (DNR-L300-D1) may be formed (S1), and after depositing Cr/Au (10 nm/100 nm), source, drain, and gate electrodes may be formed through a lift-off process (S2).

According to an embodiment, the step of forming the passivation layer (S3, S4, and S5) may include depositing the passivation layer on the electrodes, forming a masking layer, and then patterning the passivation layer by etching to expose the electrodes. For example, a passivation deposition film (e.g., a SiO₂ layer, a thickness of about 1 μm) may be deposited (S3), and a patterned masking layer (e.g., DPR-i2402) may be formed on the passivation film (S4). The masking layer may be patterned so that each electrode is exposed after the formation of the passivation layer (e.g., electrode open patterning). Next, the passivation layer may be patterned by performing reactive ion etching (RIE) on the passivation deposition film (S5).

According to an embodiment, the step of forming the conductive polymer channel layer (S6 and S7) may include forming the conductive polymer channel layer on the substrate between the source and drain electrodes, and a conductive polymer material may be coated after patterning a conductive channel region on the passivation layer. For example, the conductive channel region may be patterned with a photoresist (e.g., Photoresist (DPR-i2402)) (S7), and a conductive material (e.g., EG-PEDOT: PSS) may be drop-casted or spin-coated onto the channel region.

According to an embodiment, the step of forming the ion-selective membrane layer (S8 and S9) may include a step S8 of patterning a well after applying a polymer film (e.g., SU-8 3050) on the conductive polymer channel layer, and a step S9 of forming the ion-selective membrane layer in the well W. The conductive polymer channel layer may be exposed to a bottom surface of the well W, and the ion-selective membrane may be formed by drop casting or spin coating.

According to an embodiment, the present disclosure may provide the sensor device 1 which is an ultra-small/low-power operating wafer-level chip platform. The sensor device 1 is capable of detecting not only multiple-type ions in body fluids or saliva (e.g., detecting four types of ions in saliva) but also simultaneously detecting sugar and pH.

According to an embodiment, the present disclosure may provide a sensor (chip) (e.g., the reference numeral 20 of FIG. 1) or a sensor device (e.g., the reference numeral 1 of FIG. 1) for ultra-small and low-voltage multi-type ion detection based on an organic electronic material (e.g., an organic electrochemical transistor (e.g., OECT)) that may be utilized in a technology for a wearable platform for healthcare and a fixed multi-type ion detection platform. That is, it is manufactured in the form of an OECT, so that the sensing may be performed without a separate reference electrode and an operation of sensing and analysis.

According to an embodiment, the present disclosure may provide a sensor (chip) or sensor device for multi-type ion detection that may be introduced into a field disease-related diagnostic and preventive medical device based on body fluids (e.g., blood) and saliva. According to an embodiment, the present disclosure may provide a multi-sensor platform utilizing a small transistor form, which may enable continuous and convenient health monitoring in daily life. In addition, the present disclosure may provide a sensor (chip) or sensor device for multi-type ion detection capable of sensing and monitoring multi-type ions in water resources.

### EXAMPLES

### Preparation of DMSO-modified PEDOT: PSS

Clevios PH1000 and 20 vol % DMSO were mixed at room temperature (rt, about 15° C. to 25° C.) to obtain DMSO-modified PEDOT: PSS.

### Preparation of EG-modified PEDOT: PSS

Clevios PH1000, 5% EG, 0.1 wt % dodecyl benzene sulfonic acid, and 1 wt % (3-glycidyloxypropyl) trimethoxysilane were mixed at room temperature to obtain EG-modified PEDOT: PSS.

### Preparation of K⁺ Ionophore-containing Composition

The composition was prepared by mixing valinomycin, KTCIPB, PVC, and DOS (100 mg) in 1 mL of cyclohexanone. A mixing ratio (weight ratio) of valinomycin, KTCIPB, PVC, and DOS is 2:0.5:32.75:64.75 (100 mg).

### Preparation of Na⁺ Ionophore-containing Composition

The composition was prepared by mixing Na Ionophore X, Na-TFPB, PVC, and DOS (100 mg) in 1 mL of THF. A mixing ratios (weight ratio) of Na Ionophore X, Na-TFPB, PVC, and DOS is 1:0.55:33:65.45.

### Preparation of Ca²⁺ Ionophore-containing Composition

The composition was prepared by mixing ETH129, NaTFPB, PVC, and DOS (100 mg) in 660 uL of THF. A mixing ratio (weight ratio) of ETH129, NaTFPB, PVC, and DOS is 1:0.5:33:65.45 (100 mg).

### Preparation of Cl⁻ Ionophore-containing Composition

The composition was prepared by mixing ETH9033, TDMACl, PVC, and DOS (100 mg) in 660 μl of THF. A mixing ratio (weight ratio) of ETH9033, TDMACl, PVC, and D OS is 1.9:0.27:35.4:62.3.

### Preparation of PANi-PVC Gel-containing Composition

Aniline was prepared by mixing phytic acid (0.92l mL), aniline (0.458 mL), and DI (2 ml). 0.286g of ammonium persulfate and DI (1 mL) were mixed. PVC gel was prepared by mixing PVC (0.1667 g), dibutyl adipate (DVA) (0.834 mL), EMIM TFSI (22μL), and THF (2 mL). All prepared materials were mixed to prepare the PANi-PVC gel-containing composition.

A multi-type ion measurement sensor was manufactured according to the sensor configuration of FIG. 4 and the manufacturing method of FIG. 5. Cr/Au-based OECT electrodes were formed, and modified PEDOT: PSS was spin-coated to form conductive polymer channels. The sensor for multi-type ion and pH sensing was manufactured by drop-casting (drop coating) an ion-selective membrane layer including the ionophores and the PANi-PVC gel-containing composition described above onto a conductive polymer channel layer (i.e., EG-modified PEDOT: PSS).

### Characteristic Analysis

### (1) Evaluation of Sensitivity

An image and ion sensing characteristics were evaluated after drop casting of four ion-selective membranes.

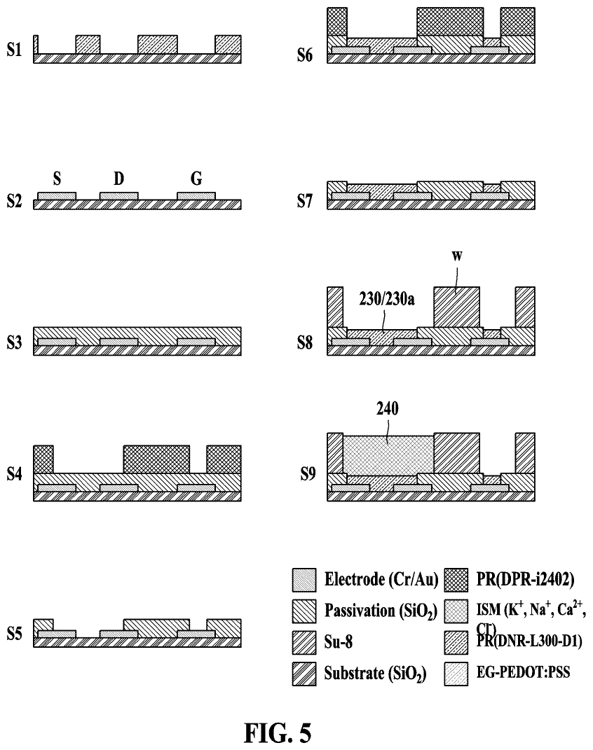

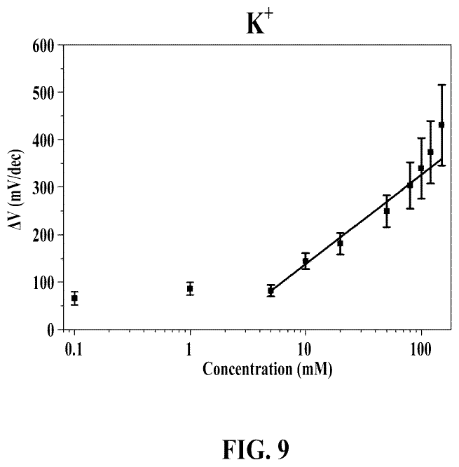

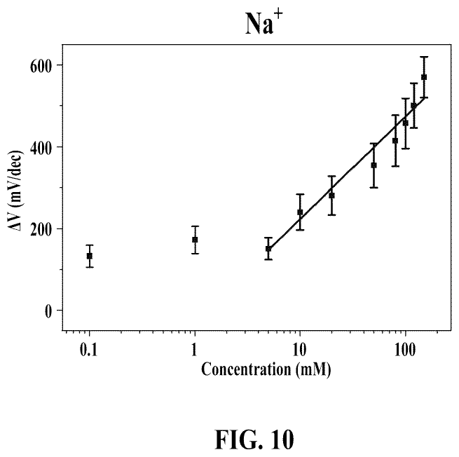

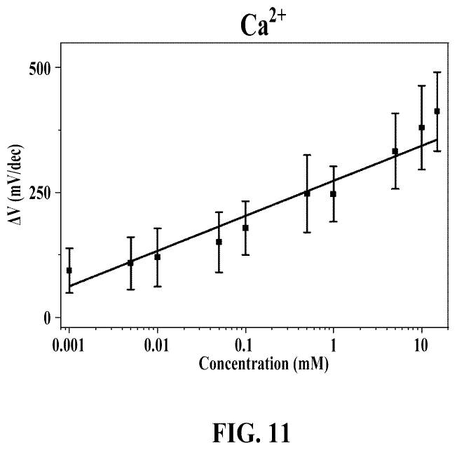

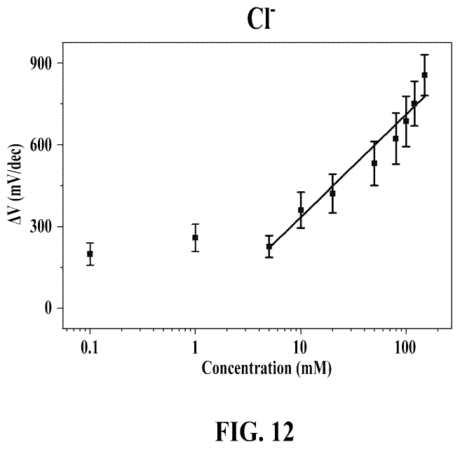

FIG. 6 illustrates an optical microscope (OM) image after forming an ion-selective membrane for four types of ions (K, Na, Ca, and Cl) in an OECT flatform. The ion sensing measurement results may be confirmed in FIGS. 9 to 12. In FIGS. 9 to 12, K⁺, Na⁺, and Cl⁻ sensing has a concentration range of 0.1 to 150 mM, and Ca²⁺sensing has a concentration range of 0.001 to 15 mM. It may be confirmed that sensing gain characteristics of K (to 187 mV/dec.), Na (to 250 mV/dec.), Ca (to 70 mV/dec.), and Cl (to 230 mV/dec.) were obtained.

The image and pH sensing characteristics were evaluated after PANi drop coating.

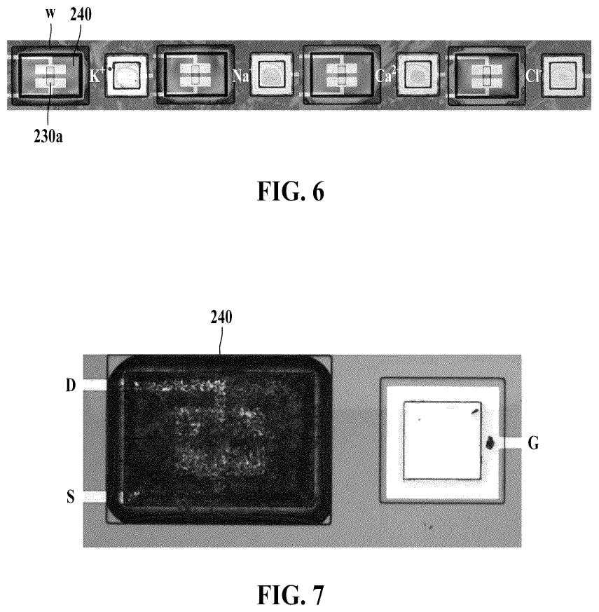

FIG. 7 illustrates an OM image after forming a PANi membrane subjected to drop casting (drop coating) for pH sensing in an OECT platform.

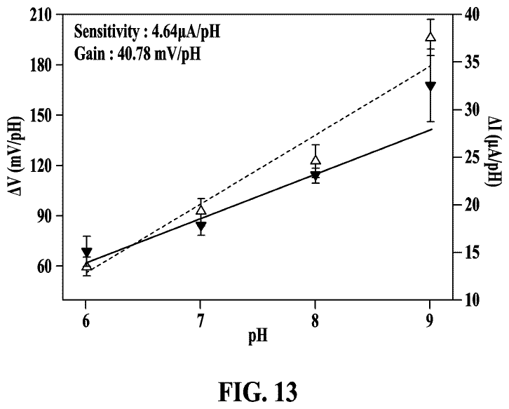

In FIG. 13, when analyzing pH sensing characteristics using the OECT scheme, a sensing signal with a sensing gain of 40.78 mV/pH may be confirmed.

An image and sugar sensing characteristics of an OECT-based sugar sensor were evaluated.

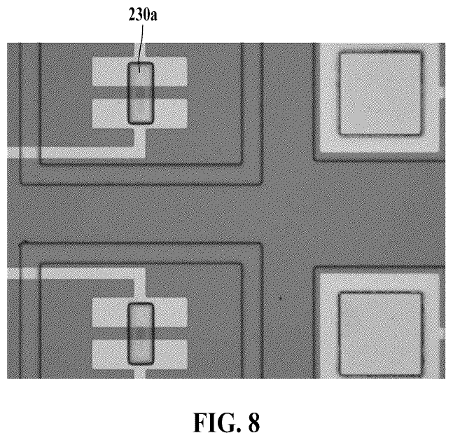

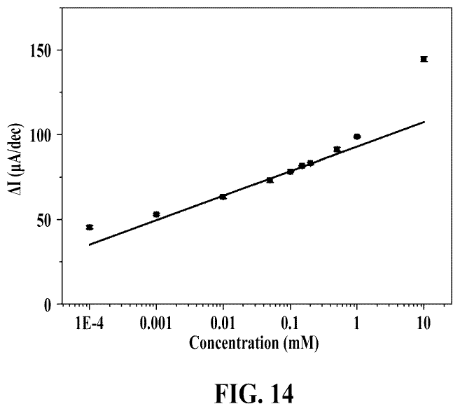

FIG. 8 illustrates an OM image of a sugar sensor manufactured with PEDOT: PSS-based OECT (EG-modified PEDOT: PSS) for blood glucose sensing in an OECT platform. In FIG. 14, it may be confirmed that blood glucose sensing using PEDOT: PSS-based OECT has a sensitivity of up to 2994 μA/dec. c² in a concentration range of 0.0001 to 10 mM. That is, the present disclosure may provide a PEDOT: PSS-based OECT capable of high-sensitivity blood glucose sensing using the OECT scheme rather than an electrochemical method of the related art.

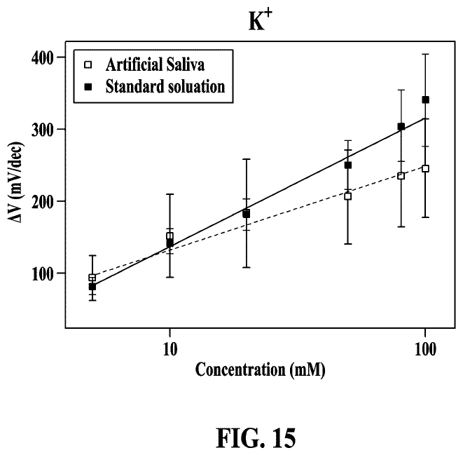

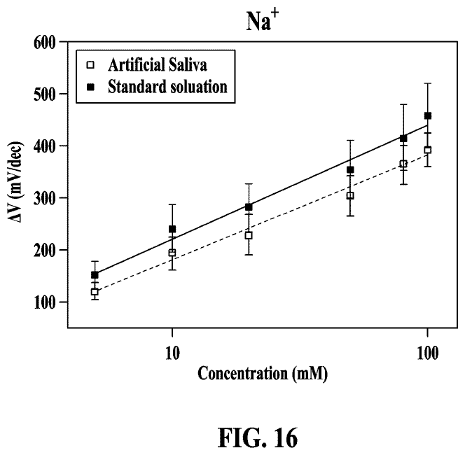

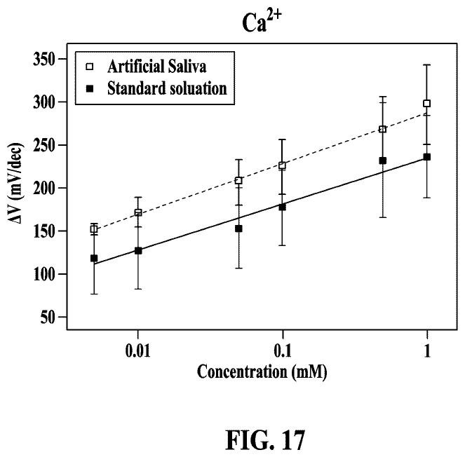

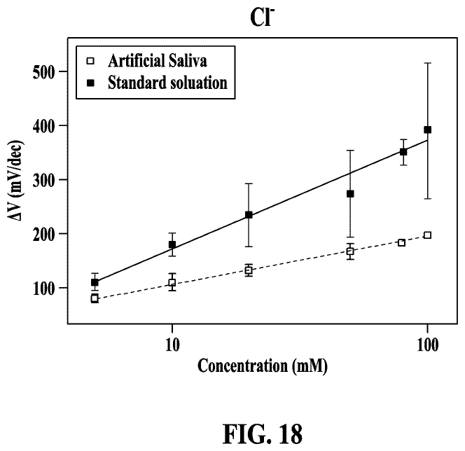

To verify the detection characteristics of multi-type ions in saliva based on the manufactured sensor chip, the sensing characteristics of multi-type ions in artificial saliva and response characteristics of multi-type ions in a standard solution according to concentration were compared. The results are shown in FIGS. 15 to 18.

It is possible to confirm the characteristic that is relatively well matched for multi-type ions in the standard solution and artificial saliva.

### (2) Evaluation of Electrical Characteristics

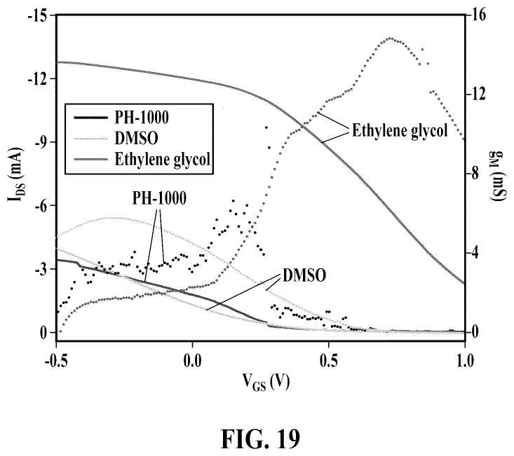

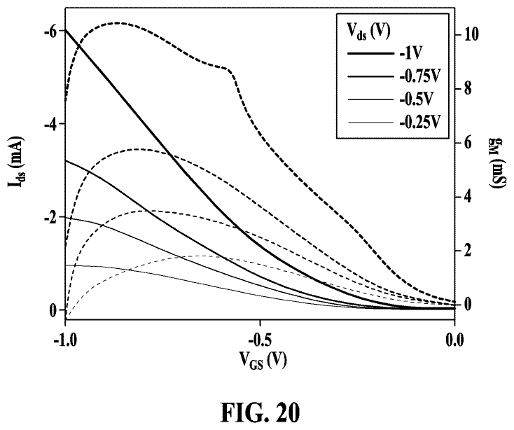

After forming a conductive polymer channel, low-voltage operation characteristics according to a channel material were evaluated. In order to improve the electrical characteristics of PH-1000, a commercially available PEDOT: PSS, the electrical characteristics were improved by utilizing the modification of DMSO and EG. In FIG. 19, gₘₐₓ of devices utilizing PH-1000, DMSO (DMSO-modified PEDOT: PSS), and EG (EG-modified PEDOT: PSS) have values of up to 5 mS, 5 mS, and up to 16 mS, respectively. In FIG. 20, the source-drain voltages of the OECT were adjusted to −1, −0.75, −0.5, and −0.25 V, confirming that the OECT (EG-modified PEDOT: PSS) may be operated at low voltage. At this time, it was confirmed that Vₜₕ was measured near −0.8V, and gₘₐₓ was 10 mS (V_{ds}: −1 V, electrolyte: 0.1 M PBS)

### (3) Surface Roughness

The RMS value of the surface roughness of EG-modified PEDOT: PSS at a thickness within 1 μm is about 2.4 nm, which is an improvement over PEDOT: PSS (about 0.8 nm).

According to an embodiment, the present disclosure may implement a chip process capable of wafer-level processing, and a sensing platform technology capable of simultaneously detecting four types of multiple ions, sugars, and pH within a single chip. In addition, it is possible to provide a multi-type ion detection sensor with an organic electronic material channel for low-power operation and a selective ion-sensitive membrane applied.

According to an embodiment, the present disclosure may provide a sensor device that is a sensor or a wafer-level chip platform capable of measuring four types of ions (K, Na, Ca, and Cl), sugar, and pH in multiple saliva within one sensor chip.

According to an embodiment, the present disclosure may implement a sensor chip array in which a thin-film gold electrode (D/S/G) array, an EG-PEDOT: PSS channel, and an ion-selective membrane are formed on a 4-inch wafer. This is not a commercial probe-type electrochemical platform of the related art, but it may be possible to provide OECT platform process technology and measurement technology based on EG-modified PEDOT: PSS channel.

A number of embodiments have been described above. Nevertheless, it should be understood that various modifications may be made to these embodiments. For example, suitable results may be achieved if the described techniques are performed in a different order and/or if components in a described system, architecture, device, or circuit are combined in a different manner and/or replaced or supplemented by other components or their equivalents. Therefore, other implementations, other embodiments, and equivalents to the claims are also within the scope of the following claims.

What is claimed is:

1. A sensor device for multi-type ion detection comprising:
a wafer substrate; and
an array of a sensor chip arranged on the wafer substrate,
wherein the sensor chip comprises a plurality of line sensors, and
the line sensor comprises:
source, drain, and gate electrodes formed on the wafer substrate; and
a sensing unit comprising a conductive polymer channel layer formed on the wafer substrate between the source and drain electrodes.

2. The sensor device of claim 1, wherein
an upper end of the conductive polymer channel layer extends to a portion of the upper end of the source and drain electrodes, and
the conductive polymer channel layer has a thickness of 1 micrometer (μm) or less.

3. The sensor device of claim 1, wherein the conductive polymer channel layer comprises dimethyl sulfoxide (DMSO)-modified poly(3,4-ethylenedioxythiophene) polystyrene sulfonate (PEDOT: PSS), ethylene glycol (EG)-modified PEDOT: PSS, or both.

4. The sensor device of claim 1, wherein
the sensor chip detects ions in a body fluid or saliva, and
the plurality of line sensors in the sensor chip has different detection targets.

5. The sensor device of claim 1, wherein the sensor chip detects at least two or more ions among K⁺ ions, Na⁺ ions, Ca⁺ ions, and Cl⁻ ions in a body fluid or saliva.

6. The sensor device of claim 1, wherein the sensor chip further comprises a function of detecting glucose in blood and a function of measuring a pH of a solution.

7. The sensor device of claim 1, wherein the sensing unit further comprises an ion-selective membrane layer formed on the conductive polymer channel layer.

8. The sensor device of claim 1, wherein
the sensing unit further comprises a well,
the conductive polymer channel layer is exposed on a bottom surface of the well, or
an ion-selective membrane layer is formed on the exposed conductive polymer channel layer.

9. The sensor device of claim 1, wherein
some of the plurality of line sensors in the sensor chip comprise the sensing unit of claim 1, and the remaining line sensors comprise the sensing unit of claim 7, or
all of the plurality of line sensors in the sensor chip comprise the sensing unit of claim 7.

10. The sensor device of claim 7, wherein
the ion-selective membrane layer comprises ionophores, and
the plurality of line sensors comprises different ionophores.

11. The sensor device of claim 10, wherein the ionophores comprise at least one of valinomycin, potassium salt of tetrakis(4-chloro-phenyl)borate (KTCIPB), Na Ionophore X, sodium tetrakis[3,5-bis(trifluoromethyl)phenyl]borate (Na-TFPB), N,N,N′,N′-tetra[cyclohexyl]diglycolic acid diamide, N,N,N′,N′-Tetracyclohexyl-3-oxapentanediamide (ETH129), NaTFPB, 3,6-didodecyloxy-4,5-dimethyl-o-phenylene-bis(mercury chloride) (ETH9033), and tridodecylmethylammonium chloride (TDMACl).

12. The sensor device of claim 10, wherein, when the ionophores comprise two or more types, a mass ratio (w/w) of one ionophore to the remaining ionophores is 1:0.1 to 3.

13. The sensor device of claim 10, wherein the ion-selective membrane layer comprises K⁺ ionophores comprising valinomycin and KTCIPB.

14. The sensor device of claim 10, wherein the ion-selective membrane layer comprises Na⁺ ionophores comprising Na Ionophore X and Na-TFPB.

15. The sensor device of claim 10, wherein the ion-selective membrane layer comprises Ca²⁺ ionophores comprising ETH129 and NaTFPB.

16. The sensor device of claim 10, wherein the ion-selective membrane layer comprises Cl⁻ ionophores comprising ETH9033 and TDMACl.

17. The sensor device of claim 10, wherein the ion-selective membrane layer comprises a polyaniline (PANi)-polyvinyl chloride (PVC) gel for pH measurement.

18. A method of manufacturing a sensor device for multi-type ion detection, the method comprising:
preparing a wafer substrate; and
forming an array of a sensor chip comprising a plurality of line sensors on the wafer substrate,
wherein the forming of the array of the sensor chip comprises:
forming source, drain, and gate electrodes on the wafer substrate; and
forming a sensing unit,
the forming of the sensing unit comprises forming a conductive polymer channel layer on the wafer substrate between the source and drain electrodes, and
the sensor device is the sensor device of claim 1.

19. The method of claim 18, wherein
the forming of the sensing unit comprises:
forming the conductive polymer channel layer on the wafer substrate between the source and drain electrodes; and
forming an ion-selective membrane layer on the conductive polymer channel layer, and
the ion-selective membrane layer is formed by drop casting or spin coating.

20. The method of claim 18, wherein
the forming of the sensing unit comprises patterning a well after applying a polymer film on the conductive polymer channel layer, and
the conductive polymer channel layer is exposed to a bottom surface of the well.
